(19) United States
(12) Patent Application Publication
(10) Pub. No.: US 2010/0152530 A1
(43) Pub. Date: Jun. 17, 2010

(54) Biocompatible Fiber Based Device for Guided Tissue Regeneration

(76) Inventors: Mark Timmer, Jersey City, NJ (US); Chunlin Yang, Belle Mead, NJ (US); Clifford G. Volpe, Hampton, NJ (US); Joseph J. Hammer, Hillsborough, NJ (US); Dhanuraj Shetty, Jersey City, NJ (US); Daniel J. Keeley, Boston, MA (US); Jackie J. Donners, West Windsor, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/335,169

(22) Filed: Dec. 15, 2008

(51) Int. Cl. A61F 2/00

(52) U.S. Cl. 600/37

(57) ABSTRACT

Tissue engineering devices for pelvic floor repair are disclosed. More specifically, tissue engineering devices made of an implant, having a central portion at least partially embedded within a nonwoven felt are disclosed.

### FIELD OF THE INVENTION

The present invention relates generally to the field of tissue repair and regeneration. More particularly, the present invention relates to devices for pelvic floor repair and methods of making the same.

### BACKGROUND OF THE INVENTION

Individuals can sometimes sustain an injury to tissue, such as musculoskeletal tissue, that requires repair by surgical intervention. Such repairs can be affected by suturing the damaged tissue, and/or by mating an implant to the damaged tissue. The implant may provide structural support to the damaged tissue, and it can serve as a substrate upon which cells can grow, thus facilitating more rapid healing.

One example of a fairly common tissue injury is damage to the pelvic floor. This is a potentially serious medical condition that may occur during childbirth or from complications thereof, which can lead to sustaining an injury of the vesicovaginal fascia. Such an injury can result in a cystocele, which is a herniation of the bladder. Similar medical conditions include rectoceles (a herniation of the rectum), enteroceles (a protrusion of the intestine through the rectovaginal or vesicovaginal pouch), and enterocystoceles (a double hernia in which both the bladder and intestine protrude). These conditions can be serious medical problems that can severely and negatively impact a patient both physiologically and psychologically.

These conditions are usually treated by surgical procedures in which the protruding organs or portions thereof are repositioned. A mesh-like patch is often used to repair the site of the protrusion.

Various known devices and techniques for treating such conditions have been described in the prior art. For example, European Patent Application No. 0 955 024 A2 describes an intravaginal set, a medical device used to contract the pelvic floor muscles and elevate the pelvic floor.

In addition, Trip et al (WO 99 16381) describe a biocompatible repair patch having a plurality of apertures formed therein, which is formed of woven, knitted, nonknitted, or braided biocompatible polymers. This patch can be coated with a variety of bioabsorbable materials as well as another material that can decrease the possibility of infection, and/or increase biocompatibility.

Other reinforcing materials are disclosed in U.S. Pat. No. 5,891,558 (Bell et al) and European Patent Application No. 0 274 898 A2 (Hinsch. Bell et al describe biopolymer foams and foam constructs that can be used in tissue repair and reconstruction. Hinsch describes an open cell, foam-like implant made from resorbable materials, which has one or more textile reinforcing elements embedded therein. Although potentially useful, the implant material is believed to lack sufficient strength and structural integrity to be effectively used as a tissue repair implant.

Despite existing technology, there continues to be a need for a tissue repair implant having sufficient structural integrity to withstand the stresses associated with implantation into the pelvic floor and also has the capability of promoting tissue ingrowth, guide tissue regeneration and enhance the integration of ingrowing tissue with scaffold.

### SUMMARY OF THE INVENTION

We have disclosed herein a tissue engineering device for pelvic floor repair. The tissue engineering device is made of an implant, where the central portion of the implant is at least partially embedded within a nonwoven felt. The nonwoven felt is located where substantial tissue ingrowth is desired.

### BRIEF DESCRIPTION OF THE FIGURES

FIG. 1 is a top plan view of one exemplary anterior implant.

FIG. 2 is a top plan view of one exemplary posterior implant.

FIG. 3 is a top plan view of another exemplary anterior.

FIG. 4 is a top plan view of another exemplary posterior implant.

FIG. 5a is a top plan view of an exemplary embodiment of an implant suitable for repair of anterior or cystocele defects with the central portion embedded in a nonwoven felt.

FIG. 5b is a top plan view of an exemplary implant suitable for repair of apical, posterior/rectocele defects with the central portion embedded in a nonwoven felt.

FIG. 5c is a top plan view of an exemplary implant suitable for repair of anterior or posterior defects with the central portion embedded in a nonwoven felt.

FIG. 6 is a top plan view of an exemplary implant for suitable for repair of anterior, posterior and/or apical defects with the central portion embedded in a nonwoven felt.

FIG. 7 an exemplary SEM micrograph of GYNEMESH PS mesh implant embedded in a 90/10 PGA/PLA nonwoven felt.

### DETAILED DESCRIPTION OF THE INVENTION

We have disclosed herein a tissue engineering device for pelvic floor repair where the tissue engineering device comprises an implant, where the central portion of the implant is at least partially embedded within a nonwoven felt.

Implants include those suitable for pelvic floor repair. Such implants include those described in U.S. Pat. No. 7,131,944 and US publication numbers US20060058575 and US20060130848 each of which is incorporated by reference herein in its entirety.

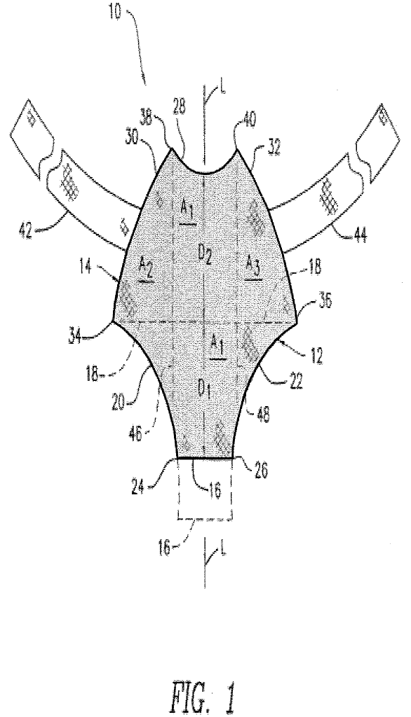

FIGS. 1-6 illustrate exemplary implants for use in pelvic floor repair. With reference to FIG. 1, an implant 10 suitable for anterior repair includes a lower portion 12 and an upper portion 14. While two distinct portions have been identified, it should be understood that the anterior implant 10 is preferably made from a single sheet of any suitable biocompatible mesh material. Accordingly, the imaginary boundaries of the various portions are indicated in FIG. 1 by dotted lines (i.e., in phantom) to facilitate consideration and discussion of the anterior implant 10.

Returning now to FIG. 1, the lower portion 12, which has a generally funnel-like shape, is demarcated by a straight lower edge 16 having a length in a range of from about 2 cm to about 5 cm, an imaginary border 18 (indicated in phantom in FIG. 1) having a length in a range of from about 8 cm to about 14 cm, and a pair of concave side edges 20, 22 having a complex (i.e., compound) arcuate shape approximating that of a female patient's pelvic anatomy. Corners 24, 26 are formed where the lower edge 16 merges with the concave side edges 20, 22, respectively. It should be noted that the lower edge 16 can be extended by as much as about 3 cm (as indicated in phantom in FIG. 1.

The distance D₁ between the lower edge 16 and the imaginary border 18 is also selected as a function of the pelvic anatomy of the patient, but typically falls within a range of from about 4 cm to about 8 cm. Of course, the nature of the mesh fabric from which the anterior implant 10 is made is such that the surgeon can modify the size and shape of the lower portion 12 to meet the needs of a particular patient. In other words, the lower portion 12 of the anterior implant 10 can be custom fitted in the surgical arena.

Still referring to FIG. 1, the upper portion 14, which has a generally dome-like shape, is demarcated by the imaginary border 18 with the lower portion 12, a curved upper edge 28 having a radius (e.g., from about 2 cm to about 4 cm) and arcuate length (e.g., from about 2 cm to about 4 cm) selected so as to avoid contact with the bladder neck of the patient, and a pair of convex side edges 30, 32 having a complex arcuate shape approximating that of the arcus tendineous fascia pelvic (ATFP). The convex side edges 30, 32 of the upper portion 14 merge with the concave side edges 20, 22, respectively, of the lower portion 12 to form corners 34, 36, respectively, while corners 38, 40 are formed where the upper edge 28 merges with the convex side edges 30, 32, respectively.

The distance D₂, as measured along the central longitudinal axis (L) of the anterior implant 10 and between the imaginary border 18 and the upper edge 28, is selected as a function of the pelvic anatomy of the patient. Typically, the distance D₂ falls within a range of from about 3 cm to about 5 cm. Like the lower portion 12, the upper portion 14 is adapted for custom fitting in the surgical arena to meet the particular needs of a patient. Thus, it should be understood that the shape and size of the upper portion 14 are subject to post-manufacture modification by the surgeon during the course of a surgical procedure.

With continuing reference to FIG. 1, strap-like implant extensions 42, 44 extend outwardly from opposite sides of the upper portion 14. More particularly, the strap-like implant extensions 42 extends laterally outward from the convex side edge 30 of the upper portion 14, while the strap-like implant extensions 44 extends laterally outward from the convex side edge 32 of the upper portion 14. The strap-like implant extensions 42, 44, whose function will be described in detail hereinafter, typically have a width in a range of from about 0.5 cm to about 2 cm, and a length in a range of from about 7 cm to about 15 cm. While the strap-like implant extensions 42, 44 preferably have a slight curvature as shown in FIG. 1, they could also extend in a linear fashion from the convex sides edges 30, 32, respectively, of the anterior implant 10. As explained previously, the surgeon can readily modify the width and length of the strap-like implant extensions 42, 44 by, for instance, cutting them with scissors or other suitable cutting instruments.

Imaginary boundary lines 46, 48, which extend generally parallel to the central longitudinal axis (L), divide the body of the anterior implant 10 into an inboard area A₁ and two outboard areas A₂, A₃ which flank the inboard area A₁. The areas A₁, A₂, and A₃ are not precise. Generally speaking, the area A₁ designates that portion of the anterior implant 10 which would function to repair a central or medial cystocele in accordance with a surgical procedure to be described in detail hereinafter, while the areas A₂, A₃ designate those portions of the anterior implant 10 which would function to repair lateral cystoceles in accordance with the same procedure.

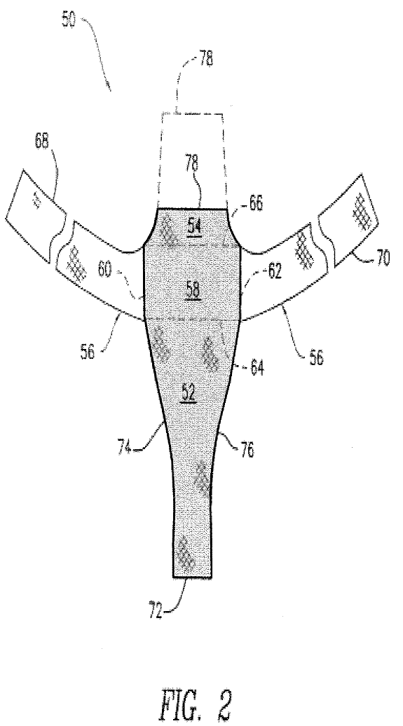

With reference now to FIG. 2, a posterior implant 50 includes a lower portion 52, an upper portion 54, and a tape portion 56. Like the anterior implant 10, the posterior implant 50 is made from a single sheet of any suitable bio-compatible mesh material. Although the posterior implant 50 is depicted in FIG. 2 as being separate and distinct from the anterior implant 10, it should be understood that the posterior implant 50 can be formed integrally with the anterior implant 10 as will be described in greater detail hereinafter. As was the case when describing the anterior implant 10 of FIG. 1, the imaginary boundaries of the various portions of the posterior implant 50 of FIG. 2 are indicated by dotted lines (i.e., in phantom) to facilitate consideration and discussion.

Returning now to FIG. 2, the tape portion 56, which is interposed between the lower portion 52 and the upper portion 54, has an imaginary central region 58 bounded by imaginary lines 60, 62, 64 and 66 and approximating the shape of a rectangle having a length of about 5 cm and a width of about 0.5 to 2 cm. The tape portion 56 also includes a pair of strap-like implant extensions 68, 70 extending outwardly from opposite ends of the imaginary central region 58. The strap-like implant extensions 68, 70, whose function will be described in greater detail hereinafter, typically have a width in a range of from about 0.5 cm to about 2 cm, a length of about 4 cm, if the strap-like implant extensions 68, 70 are attached to and terminated at the sacrospinous ligaments, or about 20 cm, if the strap-like implant extensions 68, 70 are passed through the pelvic floor via the buttocks with or without passing through the sacrospinous ligaments.

While the strap-like implant extensions 68, 70 preferably have a slight curvature as shown in FIG. 2, they could also extend in a linear fashion from opposite sides of posterior implant 50. Given the nature of the mesh material from which the posterior implant 50 is made, the width and length of the strap-like implant extensions 68, 70 can be readily modified by the surgeon to meet the needs of a particular patient.

Still referring to FIG. 2, the lower portion 52, which has a generally triangular shape, depends downwardly from the tape portion 56. More particularly, the lower portion 52 is demarcated by a straight lower edge 72 having a length in a range of from about 1.5 cm to about 3.5 cm and a pair of downwardly converging side edges 74, 76 which are either straight or slightly concave. While the length of the side edges 74, 76 is typically in a range of from about 8 cm to about 12 cm, it should be appreciated by a person skilled in the art that the physical dimensions of the lower portion 52, including the length of the side edges 74, 76, are a function of the pelvic anatomy of the patient. More particularly, the size and shape of the lower portion 52 are specifically selected for the purpose of repairing a rectocele. A person skilled in the art will also appreciate that the shape and size of the lower portion 52 are subject to post-manufacture modification by the surgeon. In cases where a rectocele repair is not required but a vaginal vault suspension is, both the lower portion 52 and the upper portion 54 can be removed from the posterior implant 50, leaving the tape portion 56 to perform the vaginal vault suspension.

With continuing reference to FIG. 2, the upper portion 54, which is demarcated at its free end by an upper edge 78 and which otherwise approximates the shape of a rectangle having a length of from about 3 cm and a width of about 1 cm, extends upwardly from the tape portion 56. The primary purpose of the upper portion 54 is to provide a means for attaching the posterior implant 50 to the anterior implant 10. Thus, for a patient who does not need the anterior implant 10, it should be appreciated that the upper portion 54 of the posterior implant 50 can be removed by the surgeon before insertion of the posterior implant 50 in such patient. It should also be appreciated that the surgeon can otherwise modify the size and shape of the upper portion 54 to meet his or her needs, such as when attaching the posterior implant 50 to the anterior implant 10. For instance, the upper edge 78 can be extended by as much as about 3 cm (as indicated in phantom in FIG. 2) to facilitate attachment of the posterior implant 50 to the anterior implant 10. Alternatively, such attachment can be facilitated by extending the lower edge 16 of the anterior implant 10 as described hereinabove.

Both the anterior implant 10 and the posterior implant 50 can be cut or punched out from a larger piece of the mesh fabrics mentioned hereinabove. If necessary, the loose ends of the severed filaments can be treated against unraveling by any suitable technique known in the art.

The anterior implant 10 and the posterior implant 50 may be provided in a variety of standard shapes and sizes (e.g., small, medium and large). After comparing these standard implants to the pelvic anatomy of a particular patient, the surgeon would select the one which best meets the patient's needs. If any modifications to the size and/or shape of the selected implant are required, they can be effected by the surgeon in the surgical arena.

The anterior implant 10 is used to make an anterior repair of a cystocele, while the posterior implant 50 is used to make a posterior repair of a rectocele. A vaginal vault suspension can be performed using the anterior implant 10 and/or the posterior implant 50. All of these treatments will be discussed in greater detail below.

What follows is a description of the two alternate embodiments referred to above and illustrated in FIGS. 10 and 11. In describing these alternate embodiments, elements which correspond to elements described above in connection with the embodiments of FIGS. 1 and 2 will be designated by corresponding reference numerals increased by one hundred. Unless otherwise specified, the alternate embodiments of FIGS. 10 and 11 are constructed and operate in the same manner as the embodiments of FIGS. 1 and 2, respectively.

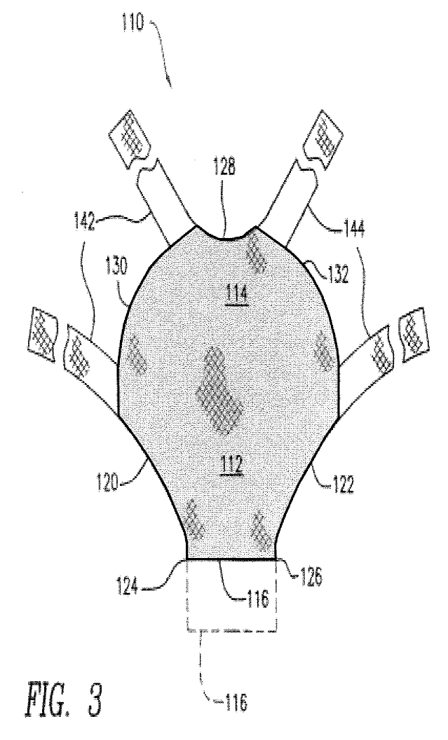

Referring to FIG. 3, there is shown an anterior implant 110 whose main difference in comparison to the anterior implant 10 of FIG. 1 involves the provision of two strap-like implant extensions 142 on one side of the anterior implant 110 and two strap-like implant extensions 144 on the opposite side of the anterior plant 110. Both of the strap-like implant extensions 142 pass through the obturator foramen on one side of a patient, while both of the strap-like implant extensions 144 pass through the obturator foramen on the other side of the patient's body. Each of the strap-like implant extensions 142 exits the patient's body through a corresponding one of two small skin incisions at the perineum (i.e., groin) on one side of the body. Similarly, each of the strap-like implant extensions 144 exits the patient's body through a corresponding one of two small skin incisions at the perineum (i.e., groin) on the other side of the body.

As compared with the anterior implant 10, the anterior implant 110 provides increased lateral support in use as a result of the provision of the extra set of strap-like implant extensions 142, 144, whose location allows the anterior implant 110 to be manufactured without the corners 34, 36 and 38, 40 which are characteristic of the anterior implant 10.

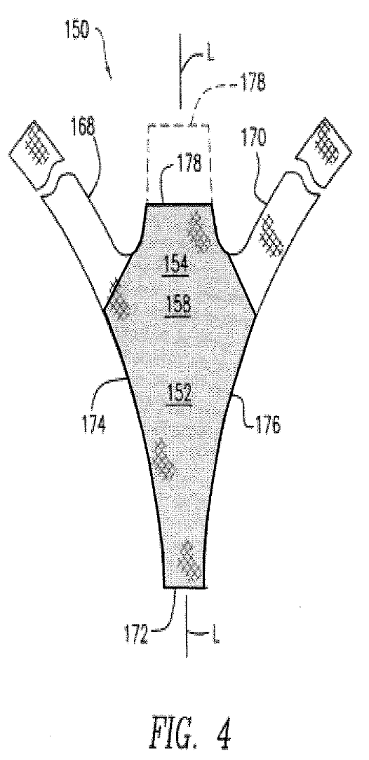

With reference to FIG. 4, there is shown a posterior implant 150 whose main difference in comparison to the posterior implant 50 of FIG. 2 involves the acute angle that strap-like implant extensions 168, 170 form with the central longitudinal axis (L) of the anterior implant 150. The angle is specifically selected so as to reduce the amount of rectal constriction in the event that the posterior implant 150 shrinks when implanted in a patient's body.

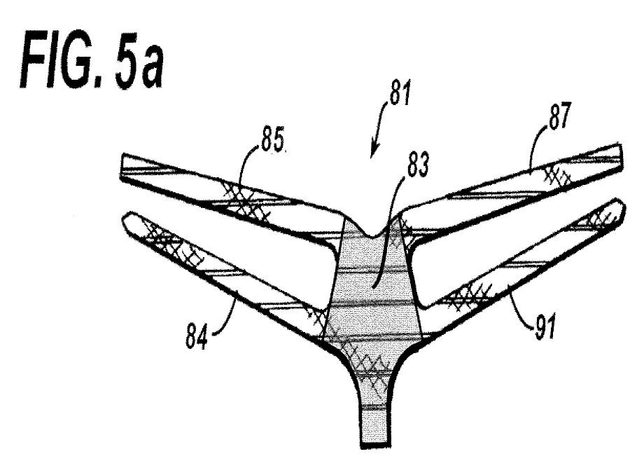

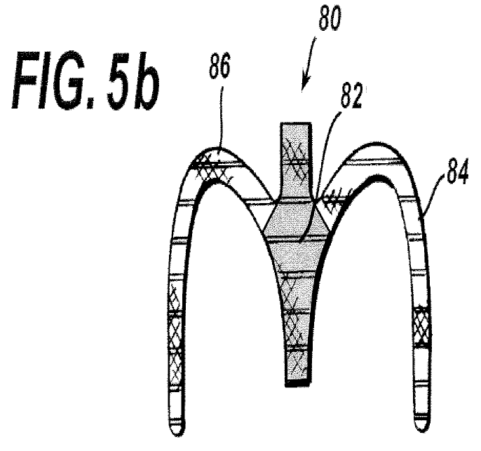

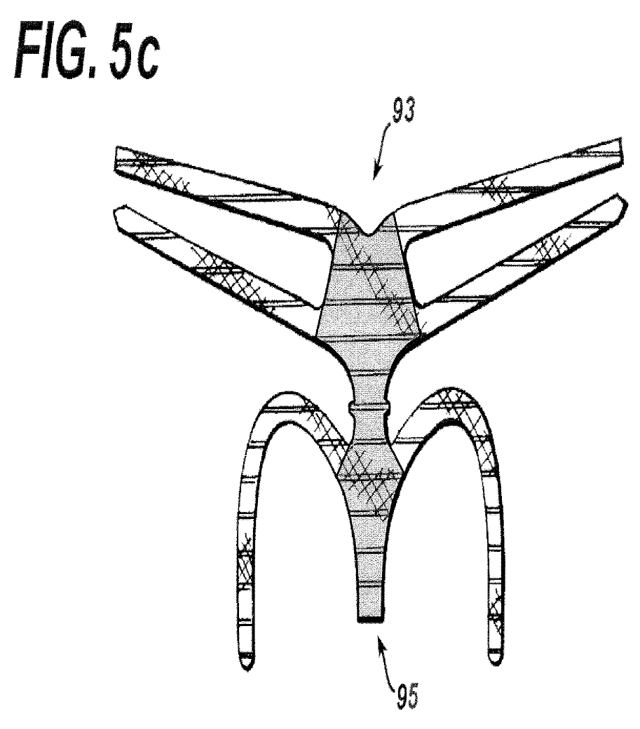

FIG. 5a illustrates a mesh suitable for use in anterior or cystocele repair that is substantially similar to implants shown in FIGS. 1 and 3. The central portion 83 of the implant 81 is designed to be placed under low or no tension between the patient's urinary bladder and vagina, to thereby reinforce the fascia in between and prevent the bladder from pushing down into the vagina. The implant 81 includes a first set of strap-like implant extensions 85, 87 and a second set of strap-like implant extensions 89, 91 extending outwardly from opposite sides of the implant. Both sets of implant extensions, 85, 87; 89, 91 are designed to pass from a location in proximity to the patient's arcus-tendineous fascia pelvis (ATFP), through the obturator membrane and out of the body through the obturator foramen. Passage of the implant extensions through tissue along the passages described above anchors the implant in place by means of friction and ultimately tissue ingrowth into the implant.

An exemplary implant for treating an apical, posterior/rectocele repair, substantially similar to implants shown in FIGS. 2 and 4, is shown in FIG. 5b. The implant 80 includes a central portion 82 that is designed to be placed under low or no tension between the patient's rectum and vagina, reinforcing the fascia there between and preventing the rectum from pushing up into the vagina. The implant 80 includes two strap-like implant extensions 84, 86 extending outwardly there from in opposite directions. These implant extensions are designed to extend through the patient's sacro-spinous ligaments and exit the body through the gluteous maximus in the vicinity of the anus.

FIG. 5c illustrates an implant that is suitable for both anterior and posterior repair. Each portion of the combination implant, the anterior portion 93 and the posterior portion 95, is substantially similar to that described above for separate implants.

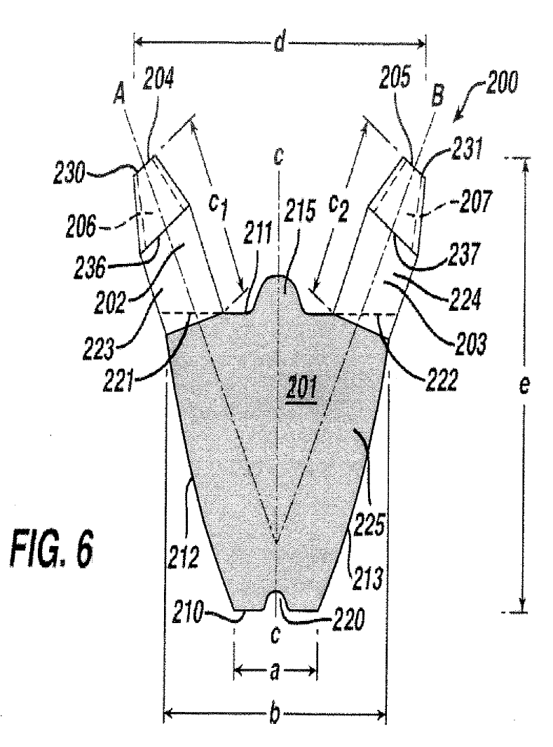

Referring now to FIG. 6, an implant 200 is provided having particular application for repair of anterior, posterior and/or apical vaginal defects.

The implant 200 has a central portion 201 having anterior and posterior edges 210, 211, and first and second lateral side edges 212, 213 that may be slightly arced as shown. The anterior edge 210 has a recess 220 extending inwardly therein and the posterior edge has a tab element 215 extending outwardly there from. The recess and tab element are both substantially centrally located along the anterior and posterior edges respectively as shown to aid in properly positioning the implant. In addition, the tab element 215 provides additional material for attachment to the uterus if desired. The central portion is preferably sized and shaped to be positioned either between the urinary bladder and the upper ⅔ of the vagina, or between the rectum and the upper ⅔ of the vagina.

The implant further has first and second 202, 203 strap-like extension portions extending outwardly from the central portion to first and second distal ends 204, 205. The strap-like extension portions extend outwardly from first and second end regions 221, 122 of the posterior edge 211 of the central body portion at an angle so as to substantially form a “Y” shaped implant in combination with the central body portion 201. In a preferred embodiment, lines A and B that substantially symmetrically bisect a top surface 223, 224 of the strap-like extension portions, and line C that substantially symmetrically bisects a top surface 225 of the central body portion, intersect within the central body portion as shown in FIG. 6.

Each of the first and second strap-like extension portions 202, 203 each further include a pocket 206, 207 at their respective distal ends. Each pocket has a closed end 230, 231 substantially adjacent to the distal ends 204, 205 of the strap-like extension portion, two closed sides, and an open end 236, 237 proximal of the closed end, with the open end opening toward the central body portion 201 as illustrated. Preferably, the first and second pockets and underlying strap-like extension taper inwardly from the open end to the closed end as shown in FIG. 6.

In a preferred embodiment, the anterior edge 210 has a length a of approximately 30 mm, and the posterior edge 211 has a length b of approximately 80 mm. Further, the strap-like extensions 202, 203 preferably have a length c1, c2 of approximately 40 mm, with the implant 200 having an overall width and length d, e of approximately 10.5 cm and 9 cm respectively.

The implants described above may be comprised of any suitable biocompatible material, absorbable or non-absorbable, synthetic or natural or combination thereof. Preferably the implant is a mesh type material, and in one embodiment, is constructed of knitted filaments of extruded polypropylene, such as that manufactured and sold by Ethicon, Inc. of Somerville, N.J. under the name GYNEMESH PS. In another embodiment, the mesh is partially absorbable and is constructed of knitted filaments of extruded polypropylene and filaments of a segmented block copolymer of glycolide and epsilon-caprolactone sold under the tradename MONOCRYL (Ethicon, Inc, Somerville, N.J.), such as that manufactured and sold by Ethicon, Inc. of Somerville, N.J. under the name ULTRAPRO. These materials are approved by the FDA in the United States for implantation into the human body for a variety of uses.

The central portion of the implants, as described above is at least partially sandwiched between two nonwoven fiber batts, the batts are subsequently entangled together leaving the central portion of the implant at least partially embedded in a nonwoven felt. The nonwoven felt is located in the central portion of the implant. The central portion of the implant is the part of the implant that supports the tissue in need of repair. The central portion of the implant 10 shown in FIG. 1 includes the upper portion 14 and lower portion 12 and the central portion of the implant 110 includes upper portion 114 and lower portion 112. The central portion of the implant 50 shown in FIG. 2 includes lower portion 52, upper portion 54, and tape portion 56, while the central portion of implant 150 includes lower portion 152, upper portion 154, and tape portion 156. The central portion of the implants shown in FIGS. 5a-5c and FIG. 6 are described above. The nonwoven felt is not located on the strap-like implant extensions of the implant. The nonwoven felt layers provide the structure needed for enhanced tissue ingrowth.

Non-woven fiber batts can be created by a variety of techniques known in the textile industry. The nonwoven fiber batt is made by processes other than spinning, weaving or knitting. For example, the nonwoven fiber batt may be prepared from yarn, scrims, netting, fibers or filaments that have been made by processes that include spinning, weaving or knitting. The yarn, scrims, netting fibers and/or filaments are crimped to enhance entanglement with each other. Such crimped yarn, scrims, netting fibers and/or filaments may then be cut into staple that is long enough to entangle. The staple may be carded to create the nonwoven fiber batts, which may be then entangled or calendared. Additionally, the staple may be kinked or piled. Other methods known for the production of nonwoven fiber batts may be utilized and include such processes as air laying, wet forming and stitch bonding.

The nonwoven fiber batt is comprised of fibers. The fibers used to make the nonwoven fiber batts can be monofilaments, yarns, threads, braids, or bundles of fibers. In any of the above structures, mechanical properties of the material can be altered by changing the density or texture of the textile, or by embedding particles in the fibers used to make the nonwoven. The density of the nonwoven fiber batts used in the invention range from 1 to 10 mg/cm², preferably between 2 and 6 mg/cm².

The fibers used to make the nonwoven fiber batt are made of biocompatible, bioabsorbable polymers. Examples of suitable biocompatible, bioabsorbable polymers include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) and blends thereof.

For the purpose of this invention, aliphatic polyesters include, but are not limited to, homopolymers and/or copolymers of monomers selected from the group consisting of lactide (which includes lactic acid, D-, L- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, and blends thereof. In one embodiment, the fibers are comprised of homopolymers and/or copolymers of monomers selected from the group consisting of lactide, glycolide, and p-dioxanone and blends thereof. In yet another embodiment, the fibers are comprised of 90/10 poly(glycolide-co-lactide) (90/10 PGA/PLA). In yet another embodiment, the fibers are comprised of 50:50 ratio of fibers of (90/10 PGA/PLA) to fibers of poly(p-dioxanone) (PDO).

The nonwoven fiber batts are combined with the implant by at least partially sandwiching the central portion of the mesh implant between two layers of non-woven batts. The nonwoven fiber batts may be attached to the implant via processes such as air entanglement, needlepunching, hydroentanglement, and the like, thereby at least partially embedding the central portion of the implant in a nonwoven felt. These processes use an air jet, needles or barbed needles, and water jet respectively to provide interlocking between the fibers of the nonwoven felts and the pelvic floor device. The jet action and the needlepunching provide further channels for tissue ingrowth.

In one embodiment, the nonwoven fiber batts are attached to the implant via needlepunching. This method entails at least partially sandwiching the central portion of the implant between two nonwoven fiber batts by placing a nonwoven fiber batt on each side of the central portion of the implant. This construct is then passed through a needlepunch machine whereby an array of barbed needles penetrate through the construct, pulling fibers and hence entangling the two layers of nonwoven fiber batts and at least partially embedding the central portion of the implant in a nonwoven felt. The number of needlepunch passes will be dependent on the final thickness, density, and resistance to delamination of the construct. For example, the number of needlepunch passes can range from about 1 to about 10. In one embodiment the preferred number of passes about 2.

In another embodiment, the nonwoven fiber batts are attached to the central portion of the mesh implant via hydroentanglement. This method entails at least partially sandwiching the central portion of the implant between two nonwoven fiber batts placed on each side of the central portion of the implant. This construct is placed on a backing mesh, then subsequently laid down on a conveyor belt or an open surface drum. The belt/drum passes the construct through a row of waterjets. The high pressurized water jets are used to push and entangle the fibers of the nonwoven felt through the mesh while the water dissipates quickly. A vacuum below the belt/drum removes the excess water. Upon passage through the waterjets, the construct is turned over for another pass through the waterjets. After entanglement, the construct is dried to remove any remaining water.

There are many process variables that will affect the thickness, density and resistance to delamination of the overall device including the water pressure, number of passes through the water jets, the backing mesh pore size, and the belt/drum speed. Water pressure can range from about 500 psi to about 3000 psi. In one embodiment, the water pressure is in the range of about 1500 psi and 2000 psi. The number of waterjet passes can range about 1 to about 10 on each side of sheet. In one embodiment the waterjet passes are in the range of about 3 to about 6 on each side. The backing mesh pore size can range from about 200×200 microns to 2000×2000 microns. In one embodiment the mesh pore size is in the range of about 400×400 microns (ASTM #40 mesh). The belt/drum speed can range between 10 ft/min to 60 ft/min. In one embodiment, the belt/drum speed is in the range of about 35 ft/min to about 45 ft/min.

The central portion of the implant is at least partially sandwiched between the nonwoven fiber batts. The partial sandwiching of the central portion of the implant between the nonwoven fiber batts can be accomplished by precutting the nonwoven fiber batts to match the area of the implant to be covered prior to attachment; by covering the entire device with the nonwoven fiber batts, attaching the nonwoven fiber batts in the desired area, and then trim away the excess nonwoven fiber batt; or attaching the nonwoven fiber batts to a sheet of mesh followed by cutting out the shape of the implant. The cutting of the nonwoven felt or mesh implant may be accomplished by laser or dye cutting or by using mechanical means such as scissors.

The total thickness of the tissue engineering devices can be between 0.5 to 3 millimeters, preferably between 0.6 to 1.2 millimeters. The total density of nonwoven in the invention can be between 50 mg/ml to 3000 mg/ml, preferably between 50 mg/ml to 300 mg/ml, and more preferably between 60 mg/ml to 90 mg/ml.

The tissue engineering device for treatment of pelvic floor repair described herein can be further enhanced by the incorporation of anti-adhesion barriers, bioactive agents, cells, minced tissue and cell lysates.

In one embodiment, the device of the present invention can be incorporated with anti-adhesion barrier(s) to prevent post-surgical tissue adhesions. Suitable anti-adhesion barriers include, but are not limited to hyaluronic acid and its derivatives; poly(ethylene glycol); oxidized regenerated cellulose, in the form of either membrane or gel; and the like.

In one embodiment, one or more bioactive agents may be incorporated within and/or applied to the tissue engineering device described herein. In one embodiment the bioactive agent is incorporated within, or coated on, the implant. In another embodiment, the bioactive agent is incorporated into the nonwoven felt layers.

Suitable bioactive agents include, but are not limited to agents that prevent infection (e.g., antimicrobial agents and antibiotics), agents that reduce inflammation (e.g., anti-inflammatory agents), agents that prevent or minimize adhesion formation, such as oxidized regenerated cellulose (e.g., INTERCEED and SURGICEL, available from Ethicon, Inc.) and hyaluronic acid, and agents that suppress the immune system (e.g., immunosuppressants) heterologous or autologous growth factors, proteins (including matrix proteins), peptides, antibodies, enzymes, platelets, platelet rich plasma, glycoproteins, hormones (e.g. estrogen creams), cytokines, glycosaminoglycans, nucleic acids, analgesics, viruses, virus particles, cell types, chemotactic agents, antibiotics, and steroidal and non-steroidal analgesics.

A viable tissue can also be included in the tissue engineering device of the present invention. The source can vary and the tissue can have a variety of configurations, however, in one embodiment the tissue is in the form of finely minced tissue fragments, which enhance the effectiveness of tissue regrowth and encourage a healing response. In another embodiment, the viable tissue can be in the form of a tissue slice or strip harvested from healthy tissue that contains viable cells capable of tissue regeneration and/or remodeling.

The tissue engineering device can also have cells incorporated therein. Suitable cell types include, but are not limited to, osteocytes, osteoblasts, osteoclasts, fibroblasts, stem cells, pluripotent cells, chondrocyte progenitors, chondrocytes, endothelial cells, macrophages, leukocytes, adipocytes, monocytes, plasma cells, mast cells, umbilical cord cells, stromal cells, mesenchymal stem cells, epithelial cells, myoblasts, tenocytes, ligament fibroblasts, neurons, bone marrow cells, synoviocytes, embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells.

The tissue engineering device can also be used in gene therapy techniques in which nucleic acids, viruses, or virus particles deliver a gene of interest, which encodes at least one gene product of interest, to specific cells or cell types. Accordingly, the bioactive agent can be a nucleic acid (e.g., DNA, RNA, or an oligonucleotide), a virus, a virus particle, or a non-viral vector. The viruses and virus particles may be, or may be derived from, DNA or RNA viruses. The gene product of interest is preferably selected from the group consisting of proteins, polypeptides, interference ribonucleic acids (iRNA) and combinations thereof.

Once the applicable nucleic acids and/or viral agents (i.e., viruses or viral particles) are incorporated into the reinforced a cellular matrix, the device can then be implanted into a particular site to elicit a type of biological response. The nucleic acid or viral agent can then be taken up by the cells and any proteins that they encode can be produced locally by the cells. In one embodiment, the nucleic acid or viral agent can be taken up by the cells within the tissue fragment of the minced tissue suspension, or, in an alternative embodiment, the nucleic acid or viral agent can be taken up by the cells in the tissue surrounding the site of the injured tissue. One skilled in the art will recognize that the protein produced can be a protein of the type noted above, or a similar protein that facilitates an enhanced capacity of the tissue to heal an injury or a disease, combat an infection, or reduce an inflammatory response. Nucleic acids can also be used to block the expression of unwanted gene product that may impact negatively on a tissue repair process or other normal biological processes. DNA, RNA and viral agents are often used to accomplish such an expression blocking function, which is also known as gene expression knock out.

The following examples are illustrative of the principles and practice of this invention, although not limited thereto. Numerous additional embodiments within the scope and spirit of the invention will become apparent to those skilled in the art once having the benefit of this disclosure.

### EXAMPLES

### Example 1

### Fabrication of Tissue Engineering Device for Pelvic Floor Repair by Needlepunching (GYNEMESH PS Mesh+90/10 PGA/PLA Nonwoven)

Nonwoven fiber batts of 90/10 (mol %) poly(glycolide-co-lactide) (90/10 PGA/PLA) fibers with a density of 2 mg/cm² were prepared at Concordia Manufacturing, LLC (Coventry, R.I.). A 15 cm×15 cm piece of GYNEMESH PS mesh (Ethicon Inc, Somerville, N.J.) was sandwiched between the nonwoven fiber batts by placing a nonwoven batt on each side of the mesh. The construct was then passed through a needlepunch loom to interlock the fiber batts and hence embedding the mesh within the nonwoven felt. Two needlepunch passes were used to generate the devices. The GYNEMESH PS Mesh+90/10 PGA/PLA nonwoven scaffolds were 1.17 mm thick with a density of 75 mg/cc.

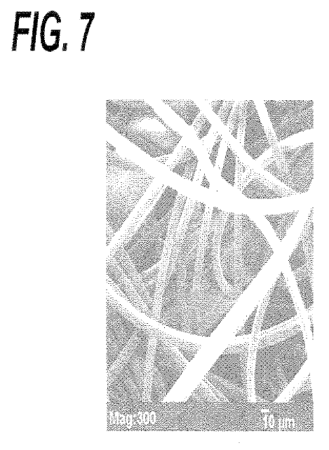

Samples were analyzed by scanning electron microscopy (SEM). The samples were mounted on a microscope stud and coated with a thin layer of gold using the EMS 550 sputter coater (Electron Microscopy Sciences, Hatfield, Pa.). SEM analysis was performed using the JEOL JSM-5900LV SEM (JEOL, Peabody, Mass.). The surface was examined for each sample. An exemplary SEM micrograph is shown in FIG. 7. The SEM shows thick polypropylene fibers of the mesh interlocked within the 90/10 PGA/PLA fibers of the nonwoven felt.

### Example 2

### Fabrication of Tissue Engineering Device for Pelvic Floor Repair by Needlepunching (ULTRAPRO Mesh+90/10 PGA/PLA Nonwoven)

Nonwoven fiber batts of 90/10 PGA/PLA fibers with a density of 2 mg/cm² were prepared at Concordia Manufacturing, LLC (Coventry, R.I.). A 15 cm×15 cm piece of ULTRAPRO mesh (Ethicon Inc, Somerville, N.J.) was sandwiched between the nonwoven fiber batts by placing a nonwoven batt on each side of the mesh. The construct was then passed through a needlepunch loom to interlock the fiber batts and hence embedding the mesh within the nonwoven felt. Two needlepunch passes were used to generate the devices. The Ultrapro mesh+90/10 PGA/PLA nonwoven scaffolds were 1.03 mm thick with a density of 71 mg/cc.

A tissue engineering device prepared as described above was cut to 4 cm×4 cm for burst testing. Burst strength of the devices was evaluated using a Mullen Burst testing apparatus. The sample was placed in the clamp zone of the testing apparatus. The clamp was activated to hold the sample in position over the rubber test diaphragm. The rubber diaphragm was pressurized with hydraulic fluid and the constantly increasing pressure caused the diaphragm to expand against the clamped mesh. The pressurization of the diaphragm continued until the sample ruptured. The pressure experienced at the point of rupture (in pound per square inch) was recorded as the burst strength.

Data represents mean±standard deviation for n=5.

| Sample | Burst Strength (psi) |
|---|---|
| ULTRAPRO Mesh + 90/10 | 130 ± 6 |
| PGA/PLA nonwoven | |
| ULTRAPRO Mesh Alone | 129 ± 4 |

### Example 3

### Fabrication of Tissue Engineering Device for Pelvic Floor Repair by Needlepunching (GYNEMESH PS Mesh+Nonwoven Fiber Batt Having a 50:50 Ratio of (90/10 PGA/PLA) Fibers:PDO Fibers)

Nonwoven fiber batts having a 50:50 ratio of 90/10 PGA/PLA fibers and polydioxanone (PDO) fibers with a density of 1 mg/cm² were prepared at Concordia Manufacturing, LLC (Coventry, R.I.). A 15 cm×15 cm piece of GYNEMESH PS mesh (Ethicon Inc, Somerville, N.J.) was sandwiched between the nonwoven fiber batts by placing a nonwoven batt on each side of the mesh. The construct was then passed through a needlepunch loom to interlock the fiber batts and hence embedding the mesh within the nonwoven felt. Two needlepunch passes were used to generate the devices. The GYNEMESH PS Mesh+(50:50) (90/10 PGA/PLA):PDO nonwoven scaffolds were 0.97 mm thick with a density of 60 mg/cc.

### Example 4

### Collagen Fibril Deposition

The scaffolds prepared as described in Example 1 were seeded with human fibroblasts following the procedure below: Die punched discs of the scaffolds (6 mm), were sterilized by ethylene oxide, and placed into wells of a 24 well low binding plate. The scaffold discs were washed with DMEM medium, seeded with human fibroblasts, and were then incubated for 21 days in a 37° C. humidified incubator in 95% air: 5% CO₂. The cell seeded discs were fixed in formalin and then embedded in paraffin.

Cross-sections were stained with Sirius Red and viewed with a polarizing microscope to show birefringence patterns of the deposited collagen. It was found that there was a robust deposition of the newly synthesized collagen fibrils that were well integrated with fibers of the non-woven scaffolds.

### Example 5

### Rabbit Fascia Repair Study

The tissue engineered devices prepared in Examples 1 and 3 were evaluated in an intrafascial model in the New Zealand White (NZW) rabbit for pelvic floor repair.

### Animal Care

The animals used in this study were handled and maintained in accordance with all applicable sections of the Final Rules of the Animal Welfare Act regulations (9 CFR), the Public Health Service Policy on Humane Care and Use of Laboratory Animals, and the Guide for the Care and Use of Laboratory Animals. The protocol and any amendments or procedures involving the care or use of animals in this study was reviewed and approved by the testing facility's Institutional Animal Care and Use Committee prior to the initiation of such procedures.

The animals were individually housed in stainless-steel cages meeting USDA regulations. A 12-hour light/12-hour dark photoperiod was maintained. Room temperature was maintained within a target range of (61-72° F.) with a target humidity of 30-70% RH. Animals were fed standard rabbit chow at least twice daily and were provided well water ad libitum.

### Materials and Methods

### Animals

All procedures were performed under aseptic conditions. Prior to surgery, the appropriate drugs were administered, and general anesthesia was induced using Ketamine. General anesthesia was maintained with isoflurane delivered in oxygen. A single midline incision at least 3 cm long was made on the dorsal surface caudal to the last rib of the animal. Two separate incisions were then created approximately 1 cm off the midline in the superficial fascia on both the right and left dorsal sides. The off-midline incision lengths were at least 3 cm. An approximate 1 cm×2 cm defect was created in the deep fascia approximately 2 to 3 cm off the midline and caudal to the last rib. The test articles (1 cm×2 cm) were placed into the defect and trimmed to size. The material was sutured in place with 5-0 PDS in a continuous pattern.

### Test Materials

The tissue engineering devices were prepared by needlepunching as described in Examples 1 and 3. Test materials were cut into 2×3 cm segments and sterilized by ethylene oxide. GYNEMESH PS, which served as a mesh-alone control, was trimmed to 2×3 cm and sterilized.

### Evaluation of Tissue Ingrowth

At 60 (±2) and 120 (±2) days post-implantation, the animals were euthanized by an intravenous overdose of sodium pentobarbital solution followed by exsanguination via severing the femoral vessels. The implant sites were then excised with a border of native tissue. Each implant was transversely bisected and the cranial end was attached to plastic material and fixed in 10% neutral buffered formalin. Following formalin fixation of the cranial end of each implant, the site was trimmed to yield one cross-section through the area with the greatest amount of implant material. Trimmed specimens were processed for paraffin embedding to yield one hematoxylin and eosin-stained slide/block.

Histopathological evaluations were performed by the pathologist. The average internodal connective tissue thickness (ICT) was measured at the approximate midpoint between the mesh nodes and limited to the collagenous portion of the connective tissue (versus vascular, adipose, and more purely cellular elements of tissue).

### Results

The average ICT at 60 and 120 days is presented below. Data represents mean±standard deviation for n=4. The symbol (*) indicates statistical difference from GYNEMESH PS control material (p<0.05, Tukey's Multiple Comparison Test).

Internodal Connective Tissue (ICT)

| Test Material | 60 days | 120 days |
|---|---|---|
| GYNEMESH PS Mesh + | 1.31 ± 0.34* | 0.79 ± 0.59 |
| 90/10 PGA/PLA non- | | |
| woven | | |
| GYNEMESH PS Mesh + | 1.23 ± 0.24* | 1.09 ± 0.38 |
| (50:50) (90/10 | | |
| PGA/PLA):PDO non- | | |
| woven | | |
| GYNEMESH PS Mesh | 0.71 ± 0.17 | 0.74 ± 0.26 |

### Conclusions

The tissue engineering devices exhibited greater tissue ingrowth than the mesh alone at 60 days. ICT within the GYNEMESH PS Mesh+90/10 PGA/PLA nonwoven was similar to the mesh alone at 120 days. Differences in ICT between the tissue engineering devices at 120 days may be attributed to the different degradation rates of the nonwoven materials.

### Example 6

### Fabrication of a Tissue Engineering Device for Pelvic Floor Repair by Hydroentanglement (ULTRAPRO Mesh+90/10 PGA/PLA Nonwoven)

Nonwoven fiber batts of 90/10 PGA/PLA with a density of 2 mg/cm² were fabricated at Concordia Manufacturing, LLC (Coventry, R.I.). ULTRAPRO mesh (30 cm×30 cm) was sandwiched between the nonwoven felts by placing a nonwoven felt on each side of the mesh. This construct was placed on an ASTM#40 backing mesh and was hydroentangled at 1500 psi, drum speed 40 ft/min, for 3 passes on each side. Hydroentanglement interlocks the fiber batts and hence embedded the mesh within the nonwoven felt. The tissue engineering device samples were then dried between 2 sheets of sterile Gammawipes, blotted dry, and then blown dry with a cold air hairdryer. The samples were then stored under vacuum. The Ultrapro mesh+90/10 PGA/PLA nonwoven scaffolds were 0.68 mm thick with a density of 73 mg/cc.

The sample was cut to 4 cm×4 cm for burst testing. Burst strength testing was done as described in Example 2. Data represents mean±standard deviation for n=5.

| Sample | Burst Strength (psi) |
|---|---|
| ULTRAPRO Mesh + 90/10 | 172 ± 17 |
| PGA/PLA nonwoven | |
| ULTRAPRO Mesh | 129 ± 4 |

Tissue engineering devices of ULTRAPRO Mesh+90/10 PGA/PLA nonwoven fabricated by hydroentanglement demonstrated a greater burst strength than the ULTRAPRO mesh alone.

### Example 7

### Fabrication of a Tissue Engineering Device for Pelvic Floor Repair by Hydroentanglement (Ratio of (90/10 PGA/PLA) Fiber:PDO Fibers)

Nonwoven fiber batts having a 50:50 ratio of 90/10 PGA/PLA fibers and PDO fibers with a density of 2.6 mg/cm² were prepared at Concordia Manufacturing, LLC (Coventry, R.I.). ULTRAPRO mesh (30 cm×30 cm) was sandwiched between the nonwoven felts by placing a nonwoven felt on each side of the mesh. This construct was placed on an ASTM#40 backing mesh and was hydroentangled at 1500 psi, drum speed 40 ft/min, for 3 passes on each side. The tissue engineering device samples were then dried between 2 sheets of sterile Gammawipes, blotted dry, and then blown dry with a cold air hairdryer. The samples were then stored under vacuum. The Ultrapro mesh+(50:50) (90/10 PGA/PLA):PDO nonwoven scaffolds were 0.68 mm thick with a density of 78 mg/cc.

### Example 8

### Effect of Mesh Size, Water Pressure, and Number of Passes on Thickness and Resistance to Delamination of Hydroentangled Tissue Engineering Device

Tissue engineering devices were fabricated with ULTRAPRO mesh and 90/10 PGA/PLA nonwoven fiber batt materials as described in Example 6. Processing parameters were varied as listed in the table below. The thickness of each sample was measured with a federal gauge. Resistance to delamination of the nonwoven felt from the mesh implant was measured by initiating a 1 inch delamination in a 2 in×6 in sample and measuring the force required to propagate delamination along the sample using an mechanical tester (Instron, Norwood, Mass.). Data for thickness and resistance to delamination is presented as mean±standard deviation for n=4 in the table below.

| ULTRAPRO Mesh + | | |
|---|---|---|
| 90/10 PGA/PLA | | Resistance to |
| Nonwoven Process | | Delamination |
| Parameters | Thickness (mm) | (lbf/inch) |
| Large backing | 0.77 ± 0.06 | 1.15 ± 0.04 |
| mesh*, 1500 psi, | | |
| 6 passes | | |
| Large backing | 0.80 ± 0.10 | 0.94 ± 0.05 |
| mesh*, 1500 psi, | | |
| 3 passes | | |
| Medium backing | 0.74 ± 0.07 | 1.22 ± 0.13 |
| mesh*, 1500 psi, | | |
| 6 passes | | |
| Medium backing | 0.73 ± 0.04 | 0.81 ± 0.14 |
| mesh*, 1500 psi, | | |
| 3 passes | | |
| Fine Backing | 0.67 ± 0.05 | ** |
| mesh*, 1500 psi, | | |
| 6 passes | | |
| Fine backing | 0.65 ± 0.05 | ** |
| mesh*, 1500 psi, | | |
| 3 passes | | |
| Medium backing | 0.71 ± 0.06 | 0.23 ± 0.1 |
| mesh*, 1000 psi, | | |
| 6 passes | | |

* large, medium, and fine meshes defined with pore sizes of 2200 × 2000 microns, 800 × 600 microns, and 430 × 400 microns, respectively
** propagation of delamination did not occur-nonwoven fibers in layer pulled apart

The results showed that ULTRAPRO Mesh+90/10 PGA/PLA nonwoven scaffold fabricated by hydroentanglement with a fine backing mesh did not demonstrate any delamination of the nonwoven felt. Increasing the number of hydroentanglement passes increases the resistance to delamination. Decreasing the water pressure decreases the resistance to delamination. The use of backing meshes with smaller pore sizes decreases the thickness of the construct.

### Example 9

### Pig Fascia Repair Study

The efficacy of the tissue engineering devices prepared in Examples 6 and 7 in increasing tissue ingrowth into the mesh was evaluated in a swine fascia model. Two different tissue engineering devices having nonwoven portions with different degradation rates were tested and compared to a mesh-alone control. Devices were sutured to the fascia on the ventral side of the pre-rectus abdominis muscle and tissue ingrowth within the meshes was assessed histologically at 3 and 5 months.

### Animal Care

The animals used in this study were handled and maintained in accordance with all applicable sections of the Final Rules of the Animal Welfare Act regulations (9 CFR), the Public Health Service Policy on Humane Care and Use of Laboratory Animals, and the Guide for the Care and Use of Laboratory Animals. The protocol and any amendments or procedures involving the care or use of animals in this study was reviewed and approved by the testing facility's Institutional Animal Care and Use Committee prior to the initiation of such procedures.

Female Yucatan mini pigs were chosen for this study because they are an established species for fascia repair studies due to relative similarity in anatomy to humans and are accepted for such studies by the appropriate regulatory agencies. The surgical site, pre-rectus fascia onlay implantation, provides placement of large (6×10 cm) mesh, which is similar to clinical mesh sizes used in hernia and pelvic floor repair. The animals were individually housed in stainless-steel cages meeting USDA regulations. A 12-hour light/12-hour dark photoperiod was maintained. Room temperature was maintained within a target range of (61-72° F.) with a target humidity of 30-70% RH. Animals were fed standard pig chow (Purina Mills 5084) at least twice daily and were provided well water ad libitum.

### Materials and Methods

### Animals

All procedures were performed under aseptic conditions. Anesthesia was induced with an intramuscular mixture of 3-5 mg/kg Telazol®, 0.5 mg/kg xylazine, and 0.011 mg/kg glycopyrrolate. After induction, anesthesia was maintained by a semi-closed circuit inhalation of 1-3% isoflurane. The surgical site (abdomen) was clipped free of hair and was then scrubbed with chlorhexidine diacetate, rinsed with alcohol, dried, and painted with an aqueous iodophor solution of 1% iodine. The entire animal was covered with a drape prior to the surgical procedure. A midline skin incision was made and the skin and subcutaneous tissue was completely dissected from the anterior abdominal wall bilaterally to expose the fascia between the oblique muscles for the placement of the 6×10 cm test article (one implant per side). The implants were placed on top of the fascia and secured with interrupted dyed sutures (VICRYL* 2-0. Ethicon, Inc., Somerville, N.J.), leaving about 1-2 inches between the mesh and the midline. The subcutaneous tissue and the skin were reapproximated with an absorbable running suture (VICRYL* 2-0, Ethicon, Inc.) and a dermal adhesive (Dermabond®, Ethicon, Inc.) was applied to the skin surface. An elastic binder was placed around the abdomen for 5-7 days post-surgery.

### Test Materials

The tissue engineering devices were prepared by hydroentanglement as described in Examples 6 and 7. The constructs were scoured in sequential iso-propanol and water baths followed by vacuum drying. The constructs were cut into 6×10 cm sheets and sterilized by ethylene oxide. The mesh-alone samples were simply cut to 6×10 cm and similarly sterilized by ethylene oxide.

90/10 PGA/PLA and PDO fibers absorbed at approximately 70 and 210 days, respectively. At 3 months in vivo, the nonwoven portion of the test material from Example 6 will be totally absorbed and the test material from Example 7 will have approximately 50% of the scaffold remaining.

Evaluation of Tissue Ingrowth within Meshes

At 3 and 5 months post-surgery, pigs were euthanized with an intravenous injection of Pentobarbitol (100 mg/kg). The implantation sites were exposed and the implant was divided into quadrants. The medial-cranial quarter of each mesh was excised with the underlying muscle tissue and a border of native tissue. sections were fixed in 10% buffered formalin and trimmed longitudinally. Sections were then processed and stained with Hematoxylin and Eosin for evaluation.

The average internodal connective tissue thickness (ICT) was measured at the approximate midpoint between the mesh nodes and limited to the collagenous portion of the connective tissue (versus vascular, adipose, and more purely cellular elements of tissue). Following measurement of ICT, the thickness of the dense portion of the connective tissue was measured. The following morphology describes areas measured as “dense”: the collagen formed physically thicker bundles or clumps that tended to have a slightly younger, less well organized appearance and slightly more cellularity. The morphology of the “less dense” zones was the opposite: the collagen formed very thin bundles that tended to be separated from one another and was often highly birefringent (indicative of organization/maturity of the collagen). The percent dense connective tissue (% DCT) was calculated by dividing dense tissue thickness by ICT and presented as a percentage.

The average ICT at 3 and 5 months is presented below. Data represents mean±standard deviation for n=7. The symbol (*) indicates statistical difference from UltraPro Mesh material (p<0.05, Tukey's Multiple Comparison Test).

Internodal Connective Tissue (ICT)

| Test Material | 3 month | 5 month |
|---|---|---|
| UltraPro Mesh + | 481 ± 326 | 561 ± 190* |
| 90/10 PGA/PLA | | |
| nonwoven | | |
| UltraPro Mesh + | 443 ± 389 | 357 ± 188 |
| (50:50) (90/10 | | |
| PGA/PLA):PDO | | |
| nonwoven | | |
| UltraPro Mesh | 228 ± 119 | 179 ± 56 |

The average % DCT at 3 and 5 months is presented below. Data represents mean±standard deviation for n=7. The symbol (*) indicates statistical difference from UltraPro Mesh material (p<0.05, Tukey's Multiple Comparison Test)

Percent Dense Connective Tissue
(% DCT)

| Test Material | 3 month | 5 month |
|---|---|---|
| UltraPro Mesh + | 54 ± 26 | 79 ± 9* |
| 90/10 PGA/PLA | | |
| nonwoven | | |
| UltraPro Mesh + | 52 ± 30 | 65 ± 11 |
| (50:50) (90/10 | | |
| PGA/PLA) nonwoven | | |
| UltraPro Mesh | 38 ± 25 | 56 ± 23 |

### Conclusions

The tissue engineering devices exhibited greater tissue ingrowth than the mesh alone. Internodal tissue was thicker and denser within the tissue engineering devices at both 3 and 5 months. The scaffolds demonstrated that they could elicit more tissue ingrowth and maintain the tissue even once the nonwoven portions had fully absorbed.

### Example 10

### Fabrication of Tissue Engineering Devices for the Treatment of Pelvic Floor Prolapse

A tissue engineering device for pelvic floor repair was fabricated using hydroentanglement and a template to localize entanglement of the non-woven. A GYNEMESH PS mesh pre-cut to the shape shown in FIG. 5c was sandwiched between two 90/10 PGA/PLA batts by placing a batt on each side of the mesh. The mesh-batt construct was then sandwiched within a plastic mask that had a cut-out of the shape and was aligned to the core portion of the device (shaded area in FIG. 5c).

The configuration was then placed on a ASTM #40 backing mesh and was hydroentangled at 1500 psi, drum speed 40 ft/min, for 3 passes on each side. The template enabled exposure to the waterjets only at the core of the device to limit entanglement in that area.

Upon completion of entanglement, the mesh-reinforced non-woven scaffold was released from the template to yield the device. Alternatively, the tissue engineering device may be made in the shape shown in the other FIGS. 1-6 above.

### Example 11

### Fabrication of Shaped Mesh-Reinforced Non-Woven Scaffolds for the Treatment of Pelvic Floor Prolapse

A tissue engineering device for pelvic floor was fabricated by hydroentanglement. Two 10 cm×35 cm strips of 90/10 PGA/PLA nonwoven felts were aligned at the midpoint of a 65 cm×35 cm sheet of poliglecaprone-25/polypropylene mesh. Strips were placed on both sides of the mesh. The configuration was placed on a ASTM #40 backing mesh and was hydroentangled at 1500 psi, drum speed 40 ft/min, for 3 passes on each side. The hydroentangled sheet was then laser-cut (Keyence ML-G9300 30 Watt Laser Cutter, 30% power, 2 passes, 150 mm/s) to the shape in FIG. 1c. Alternatively, the tissue engineering device may be made in the shape shown in the other FIGS. 1-6 above.

We claim:

1. A tissue engineering device comprising an implant having a central portion at least partially embedded within a nonwoven felt.

2. The tissue engineering device of claim 1 where the nonwoven felt is comprised of two nonwoven fiber batts.

3. The tissue engineering device of claim 2 where the nonwoven fiber batts are comprised of fibers.

4. The tissue engineering device of claim 3 where the fibers are comprised of homopolymers and/or copolymers of monomers selected from the group consisting of lactide, glycolide, epsilon-caprolactone, p-dioxanone and trimethylene carbonate.

5. The tissue engineering device of claim 4 where the homopolymers and/or copolymers of monomers selected from the group consisting of lactide, glycolide, and p-dioxanone.

6. The tissue engineering device of claim 1 where the implant is suitable for anterior, posterior, and/or apical pelvic floor repair.

7. The tissue engineering device of claim 6 where the implant is suitable for anterior repair comprising a central portion, a first set of strap-like implant extensions, and a second set of implant extensions, where the first and second set of strap-like implant extensions extend outward from opposite sides of the implant.

8. The tissue engineering device of claim 6 where the implant is suitable for apical and posterior pelvic floor repair comprising a central portion and two strap-like implant extensions that extend outward from opposite sides of the implant.

9. The tissue engineering device of claim 6 where the implant is suitable for anterior, posterior, and/or apical pelvic floor repair comprising a central portion having an anterior portion comprising a first set of strap-like implant extensions, and a second set of implant extensions, where the first and second set of strap-like implant extensions extend outward from opposite sides of the implant and a posterior portion comprising two strap-like implant extensions that extend outward from opposite sides of the implant.

10. The tissue engineering device of claim 6 where the implant is suitable for anterior, posterior, and/or apical pelvic floor repair comprising a central body portion having an anterior edge, a posterior edge, and first and second lateral side edges, wherein the anterior edge has a recess extending inwardly from the anterior edge and substantially centrally located along the anterior edge, and wherein the posterior edge has a tab element extending outwardly from the posterior edge and substantially centrally located along the posterior edge;
first and second strap-like extension portions extending outwardly to first and second distal ends from first and second end regions of the posterior edge of the central body portion, said first and second strap-like extension portions extending outwardly at an angle so as to form a substantially “Y” shaped implant in combination with the central body portion, first and second pockets located at the first and second distal ends of the first and second strap-like extensions respectively, the first and second pockets each having a closed end substantially adjacent to the distal end of the strap-like extension, and having an open end proximal thereto and opening toward the central body portion.

11. A method of making a tissue engineering device comprising the steps of:
(a) Providing an implant for pelvic floor repair having a central portion;
(b) Providing two nonwoven fiber batts;
(c) Cutting the nonwoven fiber batts to the desired shape;
(d) Placing the cut nonwoven fiber batts on each side of the central portion of the implant; and
(e) Attaching the nonwoven fiber batts to the implant by hydroentanglement, thereby at least partially embedding the central portion of the implant within the nonwoven felt.

12. A method of making a tissue engineering device comprising the steps of:
(a) Providing an implant for pelvic floor repair having a central portion;
(b) Providing two nonwoven fiber batts;
(c) Cutting the nonwoven fiber batts to the desired shape;
(d) Placing the cut nonwoven fiber batts on each side of the central portion of the implant; and
(e) Attaching the nonwoven fiber batts to the implant by needlepunching, thereby at least partially embedding the central portion of the implant within the nonwoven felt.
